(19) United States
(12) Patent Application Publication
(10) Pub. No.: US 2020/0367721 A1
(43) Pub. Date: Nov. 26, 2020

(54) AUXILIARY DISPLAY SYSTEM FOR PHOTOGRAPHING DEVICE

(71) Applicant: ANKON TECHNOLOGIES CO., LTD, Wuhan (CN)

(72) Inventors: Faguang YUE, Wuhan (CN); Na ZENG, Wuhan (CN); Miao YE, Wuhan (CN); Wenjin Yuan, Wuhan (CN); Hao Zhang, Wuhan (CN); HANG ZHANG, Wuhan (CN)

(73) Assignee: ANKON TECHNOLOGIES CO., LTD, Wuhan (CN)

(21) Appl. No.: 16/882,462

(22) Filed: May 23, 2020

(30) Foreign Application Priority Data
May 23, 2019 (CN) 201910434452.3

(51) Int. Cl. A61B 1/00; A61B 1/04; G06T 7/00; G06T 11/60

(52) U.S. Cl. CPC A61B 1/00045; A61B 1/00055; G06T 2207/10068; G06T 7/0012; G06T 11/60; A61B 1/041

(57) ABSTRACT

An auxiliary display system for a photographing device is provided. The photographing device takes images in vivo and generates image information. The auxiliary display system includes a server and a client in communication with the server. The server sequentially identifies and analyzes the image information in real time and generates suspected lesion information. The client receives suspected lesion information transmitted from the server, obtains image information corresponding to the suspected lesion information, and simultaneously displays the corresponding image information and the suspected lesion information in real time.

### CROSS-REFERENCE OF RELATED APPLICATIONS

The application claims priority to Chinese Patent Application No. 201910434452.3 filed on May 23, 2019, the contents of which are incorporated by reference herein.

### FIELD OF INVENTION

The present invention relates to a medical device, and more particularly to an auxiliary display system for a photographing device.

### BACKGROUND

In the current medical environment, when an operator examines a subject internally using a photographing device, it is often necessary to examine all regions of the subject in their entirety and to take more valid images from multiple angles in certain regions such as those with suspected lesions.

For example, in a capsule endoscopy procedure, the capsule endoscope enters the digestive tract of the subject by swallowing and then takes images. The images can be transmitted in real time by single image at a time, or all images taken in the examination can be transmitted at a time and then transferred as a whole. Real-time image display after data transmission facilitates review by the operator.

However, in the existing real-time transmission and image display system, only the images taken by the capsule endoscope can be seen. The operator can only judge from experience whether there are lesions in the captured images, while the validity of the captured images depends entirely on on-site judgment of the operator. Therefore, the operator is required to be more skilled, and non-standard operation can easily lead to misdiagnosis.

Therefore, it is necessary to design a new auxiliary display system for photographing device to facilitate judgment of the operator.

### SUMMARY OF THE INVENTION

To solve one of the above problems, the present invention provides an auxiliary display system for a photographing device, the photographing device photographs in vivo and generates image information. The auxiliary display system for photographing device comprises a server and a client in communication with the client.

The server comprises an algorithm analysis module, wherein the algorithm analysis module sequentially identifies and analyzes the image information in real time and generates suspected lesion information.

The client comprises a display control module, wherein the display control module receives suspected lesion information transmitted from the algorithm analysis module, obtains image information corresponding to the suspected lesion information, and simultaneously displays the corresponding image information and the suspected lesion information in real time.

In one embodiment, the client further communicates with the photographing device to obtain image information in real time and sends the image information to the algorithm analysis module of the server.

In another embodiment, the server further comprises a server networking detection module, and the client further comprises a client networking detection module, wherein the client networking detection module communicates with the server networking detection module.

In another embodiment, the display control module comprises a quality control module for receiving single image information in real time, and sending the image information to the algorithm analysis module for analysis, and the suspected lesion information comprises digestive tract regions, suspected lesion types, suspected lesion heat maps, an external operating time, the number of digestive tract images, and suspected lesion contour position coordinates of the current image.

In another embodiment, the quality control module analyzes the image information, the suspected lesion heat maps, the digestive tract regions, the suspected lesion types, and the suspected lesion contour position coordinates, and generates suspected lesion image information, and the quality control module further comprises a real-time quality display area for displaying the suspected lesion image information.

In another embodiment, the quality control module analyzes the number of digestive tract images and generates complete information of digestive tract regions examination, and the quality control module further comprises an examination completeness display area for displaying the complete information of digestive tract regions examination.

In another embodiment, the quality control module analyzes the external operating time and the number of digestive tract images, and generates a total examination time, examined regions and unexamined regions, and the quality control module further comprises a quality control statistics display area for displaying the total examination time, the examined regions and the unexamined regions.

In another embodiment, the display control module comprises an auxiliary image review module for receiving a plurality of images at a time and sending the plurality of images to the algorithm analysis module, wherein the plurality of images are arranged according to timestamp, and the suspected lesion information includes a digestive tract segmentation index number, a suspected lesion index number, a suspected lesion type and a lesion frame.

In another embodiment, the auxiliary image review module analyzes the digestive tract segmentation index number and generates a progress bar according to the digestive tract index number, and the auxiliary image review module further comprises a digestive tract segmentation display area for displaying the progress bar and corresponding image information.

In another embodiment, the auxiliary image review module analyzes the suspected lesion index number and generates a suspected lesion index, and the auxiliary image review module further comprises a suspected lesion index display area for displaying the suspected lesion index on the progress bar.

In another embodiment, the auxiliary image review module analyzes the image information, the suspected lesion index number, the suspected lesion type, and suspected lesion contour position coordinates, and generates suspected lesion image information, and the auxiliary image review module further comprises a suspected lesion label display area for displaying the suspected image information lesion.

Compared to the prior art, in the above method, after the algorithm analysis module analyzes and generates the suspected lesion information in real time, the display control module of the client obtains the suspected lesion information in real time, and displays the corresponding suspected lesion information and image information simultaneously. While obtaining the image information, the suspected lesion information corresponding to the image information can also be displayed, so that the operator can judge the image information based on the suspected lesion information, reduce misdiagnosis and improve the efficiency of the examination.

### BRIEF DESCRIPTION OF THE DRAWINGS

FIG. 1 shows a schematic view of an auxiliary display system for a photographing device according to the present invention.

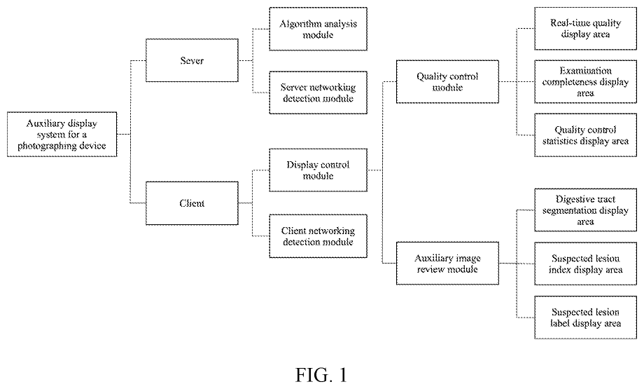

### DETAILED DESCRIPTION

In order to enable those skilled in the art to better understand the technical solutions disclosed, the present invention can be described in detail below with reference to the accompanying drawings and preferred embodiments. However, the embodiments are not intended to limit the invention, and obviously, the described embodiments are only a part of the embodiments of the present invention, but not all of them. All other embodiments obtained by those having ordinary skill in the art without creative work based on the embodiments of the present invention are included in the scope of the present invention.

The present invention provides an auxiliary display system for a photographing device, the photographing device takes images in vivo and generates image information. In the embodiment, the photographing device refers to a capsule endoscope, the capsule endoscope can be swallowed by a subject, and move with the peristalsis of the digestive tract of the subject. While moving, the capsule endoscope takes images of the digestive tract and generates a plurality of image information.

Specifically, the auxiliary display system for the photographing device comprises a server and a client.

The server comprises an algorithm analysis module, the algorithm analysis module sequentially identifies and analyzes the image information in real time and generates suspected lesion information.

The client, in communication with the server, comprises a display control module. The display control module receives suspected lesion information transmitted from the algorithm analysis module, obtains image information corresponding to the suspected lesion information, and simultaneously displays the corresponding image information and the suspected lesion information in real time.

Therefore, after the algorithm analysis module analyzes and generates the real-time suspected lesion information, the display control module of the client obtains the suspected lesion information in real time, and displays the corresponding suspected lesion information and image information simultaneously. While obtaining the image information, the suspected lesion information corresponding to the image information can also be displayed, so that the operator can judge the image information based on the suspected lesion information, reduce misdiagnosis and improve the efficiency of the examination.

In addition, the server can be set up in cloud and connected to the client via network communication, thus reducing the workload of the client. In the embodiment, the client further communicates with the photographing device to obtain image information in real time and sends the image information to the algorithm analysis module. Thus, the client acts as a communication relay between the algorithm analysis module and the photographing device. If the server directly communicates with the photographing device and receives the image information transmitted from the photographing device, the object of the present invention can also be achieved. In the embodiment, the client configured as a communication relay is also able to save image information in advance to prevent loss of image information.

The server further comprises a server networking detection module, and the client further comprises a client networking detection module, wherein the client networking detection module communicates with the server networking detection module. Thus, the client communicates with the server by the connection between the client networking detection module and the server networking detection module.

Specifically, as the client starts, the client networking detection module starts to search for the server networking detection module and carries out networking. If networking succeeds, the client can communicate with the server. If networking fails, the client networking detection module sends a broadcast every N seconds, and detects the server networking detection module until receiving an unicast signal from the server networking detection module, and at this point, the client is automatically connected to the server, otherwise the process continues to cycle. At the same time, if the client is working while the server is still off, the client can send a remote wake up broadcast and perform a remote wake up to start the server. In the embodiment, the network communication protocols of the client networking detection module and the server networking detection module support different levels such as UDP/TCP/MAC, and the network connection methods comprise Gigabit Ethernet, wireless network, or 4G Network, etc.

Further, specifically, in the embodiment, the display control module can also have different display modes, as can be specifically analyzed below. Furthermore, the display control module can select different display modes for separate display, or simultaneous display.

In the first embodiment, the display control module comprises a quality control module that receives single image information in real time, and sends the image information to the algorithm analysis module for analysis. That is, in the first embodiment, the photographing device transmits the generated image information instantaneously to the quality control module in the photographing process, so that the quality control module also receives a single image at a time. In addition, the quality control module also transmits a single image to the algorithm analysis module at a time, which identifies and analyzes each image, and then generates the suspected lesion information for each image in turn.

The suspected lesion information comprises digestive tract regions, suspected lesion types, suspected lesion heat maps, an external operating time, the number of digestive tract images, and suspected lesion contour position coordinates.

Wherein, the digestive tract comprises a plurality of regions, including specifically esophagus, stomach, small intestine, large intestine, and the difference between the regions is large, and the distance is also long. So the first judgment goes to the digestive tract region, that is to identify the image information for the digestive tract region where it is taken. Secondly, the algorithm analysis module identifies the suspected lesion type in the image through analysis to facilitate further judgment by the operator. The suspected lesion heat map can differentiate and display by the different colors the scope of the region where the suspected lesion is. Suspected lesion contour position coordinates are the position coordinates of the suspected lesion in the image information calculated by the peripheral contour of the suspected lesion heat map. The above suspected lesions information can be displayed directly or indirectly on the quality control module.

The quality control module analyzes the image information, the suspected lesion heat maps, the digestive tract regions, the suspected lesion types and the suspected lesion contour position coordinates, and generates suspected lesion image information. The quality control module further comprises a real-time quality display area for displaying the suspected lesion image information.

Specifically, the image information is displayed in the suspected lesion image information, and both the suspected lesion type and the digestive tract region are superimposed and displayed in the image information. In addition, since the suspected lesion type and the digestive tract region are displayed in text, the display text of the suspected lesion type and the digestive tract region can change automatically according to the background color of the image information for easy reading. Specifically, the color of display area of the suspected lesion type and the digestive tract region in the image information is first extracted, and then the color is reversed to form a reverse color, so that the suspected lesion type and the digestive tract region are displayed by the reverse color. As a result, the operator has a clear understanding of the results of the algorithm analysis module and can use them as a reference.

The quality control module further analyzes the suspected lesion contour position coordinates and connects the suspected lesion contour position coordinates in a line or roughly calculates to generate a lesion frame, which is superimposed in the corresponding image information.

The suspected lesion heat map does not overlap with image information and is displayed in other area of adjacent image information.

Accordingly, by the method, the real-time quality display area can display the suspected lesion image information, and by observing the suspected lesion image information in the display control module, the operator can directly observe the image information captured by the photographing device and the corresponding suspected lesion information in real time, so as to realize real-time examination.

The quality control module analyzes the number of digestive tract images and generates complete information of digestive tract regions examination, and the quality control module further comprises an examination completeness display area for displaying the complete information of digestive tract regions examination.

Specifically, the examination completeness display area presents a gastrointestinal model, on which the digestive tract regions are displayed. When the number of images of a certain digestive tract region is greater than a set threshold, the digestive tract region is determined as completely examined, and the digestive tract region is displayed in a different color on the gastrointestinal model to distinguish it from other digestive tract regions. In the embodiment, if a particular digestive tract region is determined as completely examined, the digestive tract region on the gastrointestinal model of the examination completeness display area is illuminated to be bright, while other digestive tract regions that are not examined completely are dark. This examination completeness display area can also directly display the number of images of different digestive tract regions.

The quality control module analyzes the external operating time and the number of digestive tract images, and generates a total examination time, examined regions and unexamined regions. The quality control module further comprises a quality control statistics display area for displaying the total examination time, the examined regions and the unexamined regions, so that the quality control statistics display area displays the examination progress in real time to facilitate the operator to judge the next step of the operation.

Therefore, through the different display areas on the quality control module, the operator can understand the current suspected lesion image information displayed in the real-time quality display area, the completeness information displayed in the examination completeness display area, and the examined regions displayed in the quality control statistics display area. Accordingly, the operator can not only observe the image information captured by the photographing device in real-time, but also can quickly identify the suspected lesion information in the image information, and also can understand the photographing progress. This method further saves time of the operator, and the information finally displayed on the quality control module is sufficient for the operator to make a judgment on the actual lesion.

In the second embodiment, the display control module comprises an auxiliary image review module for receiving a plurality of images at a time and sending the plurality of images to the algorithm analysis module, wherein the plurality of images are arranged according to timestamp. Thus, in the second embodiment, the photographing device transmits all or part of the image information of the subject to the auxiliary image review module immediately after the photographing is completed or partly completed, so that the auxiliary image review module receives the plurality of images at a time and sends the plurality of images to the algorithm analysis module at a time. Then the algorithm analysis module performs an overall and separate identification and analysis of the plurality of images to obtain suspected lesion information for each image.

The suspected lesion information comprises a digestive tract segmentation index number, a suspected lesion index number, a suspected lesion type, and a lesion frame corresponding to the image information.

The auxiliary image review module analyzes the digestive tract segmentation index number, and generates a progress bar according to the digestive tract segmentation index number. The auxiliary image review module further comprises a digestive tract segmentation display area for displaying the progress bar and corresponding image information.

Specifically, in the embodiment, since the plurality of images are arranged according to timestamp, and the digestive tract segmentation index numbers are also arranged according to timestamp. The progress bar is the sequence of arrangement of the digestive tract segmentation index numbers according to timestamp, and each segment or point on the progress bar is represented as the information of an image. Also, in the embodiment, the image information is displayed in the form of a thumbnail, and the thumbnail and the corresponding segment or point on the progress bar are connected to each other in a line for visual display.

The auxiliary image review module analyzes the suspected lesion index number and generates a suspected lesion index. The auxiliary image review module further comprises a suspected lesion index display area for displaying the suspected lesion index on the progress bar.

The suspected lesion index display area is labeled on the progress bar only according to the suspected lesion index number, so as to facilitate the operator to select the image information with suspected lesion, and thus also to facilitate observation of the operator. In practice, if the suspected lesion index is selected, when the progress bar jumps next time, it also jumps among other suspected lesion indexes, improving the efficiency of search and examination of suspected lesions. Therefore, the progress bar arranges the image information and the suspected lesion image information in order, and optionally jumps in the image information or the suspected lesion image information.

The auxiliary image review module analyzes the image information, the suspected lesion index number, the suspected lesion type, and the suspected lesion contour position coordinates, and generates suspected lesion image information. The auxiliary image review module further comprises a suspected lesion label display area for displaying the suspected lesion image information. The suspected lesion image information is also image information.

The auxiliary image review module extracts the corresponding image information from the plurality of images by means of the suspected lesion index numbers, and combines the suspected lesion type and the suspected lesion contour position coordinates to generate the suspected lesion image information, and displays it in the suspected lesion label display area. In addition, the image information or the suspected lesion image information can be observed in sequence through the progress bar. As described above, the suspected lesion contour position coordinates are calculated by the auxiliary image review module to form the lesion frame. The lesion frame is similarly superimposed on the image information to form the suspected lesion image information, which is not repeated here.

To sum up, the auxiliary display system for photographing device comprises a server and a client, wherein the server and the client can communicate with each other. When the photographing device obtains the image information, the display control module of the client can display the image information while displaying the identification results of the algorithm analysis module in real time. Therefore, the operator can observe the suspected lesion information in real time during the examination or after the examination is completed, thereby improving the efficiency of the operator. Further, two embodiments are provided to separately identify and calculate single image information and a plurality of images information, so as to output corresponding identification results to facilitate the examination by the operator for different cases.

It should be understood that, although the specification is described in terms of embodiments, not every embodiment merely includes an independent technical solution. Those skilled in the art should have the specification as a whole, and the technical solutions in each embodiment may also be combined as appropriate to form other embodiments that can be understood by those skilled in the art.

The present invention by no means is limited to the preferred embodiments described above. On the contrary, many modifications and variations are possible within the scope of the appended claims.

1. An auxiliary display system for a photographing device, the photographing device takes images in vivo and generates image information, wherein the auxiliary display system comprises a server and a client in communication with the server, wherein
the server comprises an algorithm analysis module, wherein the algorithm analysis module sequentially identifies and analyzes the image information in real time, and generates suspected lesion information;
the client comprises a display control module, wherein the display control module receives the suspected lesion information transmitted from the algorithm analysis module, obtains image information corresponding to the suspected lesion information, and simultaneously displays the corresponding image information and the suspected lesion information in real time.

2. The auxiliary display system of claim 1, wherein the client further communicates with the photographing device to obtain image information in real time and sends the image information to the algorithm analysis module of the server.

3. The auxiliary display system of claim 1, wherein the server further comprises a server networking detection module, and the client further comprises a client networking detection module, wherein the client networking detection module communicates with the server networking detection module.

4. The auxiliary display system of claim 1, wherein the display control module comprises a quality control module for receiving single image information in real time, and sending the image information to the algorithm analysis module for analysis; and wherein the suspected lesion information comprises digestive tract regions, suspected lesion types, suspected lesion heat maps, an external operating time, the number of digestive tract images, and suspected lesion contour position coordinates of the current image.

5. The auxiliary display system of claim 4, wherein the quality control module analyzes the image information, the suspected lesion heat maps, the digestive tract regions, the suspected lesion types, and the suspected lesion contour position coordinates, and generates suspected lesion image information; and wherein the quality control module further comprises a real-time quality display area for displaying the suspected lesion image information.

6. The auxiliary display system of claim 4, wherein the quality control module analyzes the number of digestive tract images, and generates complete information of digestive tract regions examination; and wherein the quality control module further comprises an examination completeness display area for displaying the complete information of digestive tract regions examination.

7. The auxiliary display system of claim 4, wherein the quality control module analyzes the external operating time and the number of digestive tract images, and generates a total examination time, examined regions and unexamined regions; and wherein the quality control module further comprises a quality control statistics display area for displaying the total examination time, the examined regions and the unexamined regions.

8. The auxiliary display system of claim 1, wherein the display control module comprises an auxiliary image review module for receiving a plurality of images at a time, and sending the plurality of images to the algorithm analysis module, the plurality of images being arranged according to timestamp; and wherein the suspected lesion information includes a digestive tract segmentation index number, a suspected lesion index number, a suspected lesion type and a lesion frame.

9. The auxiliary display system of claim 8, wherein the auxiliary image review module analyzes the digestive tract segmentation index number, and generates a progress bar according to the digestive tract segmentation index number; and wherein the auxiliary image review module further comprises a digestive tract segmentation display area for displaying the progress bar and corresponding image information.

10. The auxiliary display system of claim 9, wherein the auxiliary image review module analyzes the suspected lesion index number, and generates a suspected lesion index; and wherein the auxiliary image review module further comprises a suspected lesion index display area for displaying the suspected lesion index on the progress bar.

11. The auxiliary display system of claim 9, wherein the auxiliary image review module analyzes the image information, the suspected lesion index number, the suspected lesion type, and the suspected lesion contour position coordinates, and generates suspected lesion image information; and wherein the auxiliary image review module further comprises a suspected lesion label display area for displaying the suspected lesion image information.
